(19) United States
(12) Patent Application Publication
(10) Pub. No.: US 2017/0224541 A1
(43) Pub. Date: Aug. 10, 2017

(54) METHOD FOR MAKING ABSORBENT SANITARY ARTICLES

(71) Applicant: G.D S.p.A., Bologna (IT)

(72) Inventors: Paolo ARDUINI, Casalecchio di Reno (Bologna) (IT); Stefano NEGRINI, Calderara di Reno (Bologna) (IT)

(21) Appl. No.: 15/502,257

(22) PCT Filed: Jun. 25, 2015
(86) PCT No.: PCT/IB2015/054791

§ 371 Date: Feb. 7, 2017

(30) Foreign Application Priority Data
Aug. 8, 2014 (IT) BO2014A000453

(51) Int. Cl. A61F 13/15

(52) U.S. Cl. CPC A61F 13/15756; A61F 13/15723; A61F 13/15739

(57) ABSTRACT

A method for making absorbent sanitary articles including the steps of preparing at least a first continuous web and a second continuous web of material, the first having two longitudinal edges parallel to each other, cutting the first web in such a way as to divide it into a plurality of portions positioned one after the other, rotation and/or translation of at least part of the portions so as to associate them in pairs to define a plurality of pairs of portions and connecting the portions to the second web. The cutting step is performed in such a way as to form, on the first web, an alternating succession of first, second, third and fourth portions, having respective orientations different from each other and each equipped with a first and a second side parallel with each other, defining part of the longitudinal edges of the first web.

### TECHNICAL FIELD

This invention relates to a method for making absorbent sanitary articles.

The invention is therefore applicable in particular to the sector of automatic machines, and in particular for the making of absorbent sanitary articles such as disposable nappies or incontinence pads, of the disposable type, for children or adults.

### BACKGROUND ART

Various methods are known in the prior art for making absorbent sanitary articles, and in particular for joining separate accessory elements, such as, for example, closing flaps, to the main portion, that is, the absorbent portion (or core) of the article.

It should be noted that, in recent years, the development of the technology has increasingly led to the design of production lines which are able to reduce to the maximum extent the waste material which is obtained following the numerous cutting actions performed during the processing.

In this regard, numerous patent publications describe processes which, starting from two or more webs of elastic material, make cuts such as to define pieces of web having geometrical shapes which are interpenetrating with alternating orientations.

The pieces can therefore be applied directly to the supporting web (chassis) following suitable rotations or translations, avoiding the production of waste and also allowing the “leg opening” of the absorbent article to be obtained.

For example, patent document EP1941853 describes a method for making absorbent sanitary articles wherein the making of the flaps is performed starting from a pair of parallel webs of elastic material, which are cut in such a way that each of them is divided into an alternating succession of first and second pieces trapezoidal in shape.

More specifically, the pieces of the first web are specular to the pieces of the second web.

The first pieces of both the webs have the larger bases facing each other, whilst in the second pieces the smaller bases are facing each other.

In order to apply the flaps in the correct direction (that is, with the larger bases facing each other), the method comprises performing a rotation by 180° for each of the second pieces of both the webs, so as to obtain an ordered succession of pairs of pieces having a same orientation, such as to allow a direct application of the pieces, that is to say, the flaps, to the continuous supporting web.

A further solution is known from patent document EP1994919, wherein the two webs are cut in an equivalent fashion, not specular, that is to say, in such a way that each smaller base of a piece of the first web faces the larger base of a corresponding piece of the second web.

In other words, each web has a plurality of first pieces, having the larger base oriented towards the other web, and a plurality of second pieces having the smaller base oriented towards the other web.

In order to obtain a correct orientation of the flaps, the method comprises a step of rotating all the second pieces of both the webs, which unlike the method described above are alternating, but which, once rotated, similarly define an ordered succession of pairs of pieces having a same orientation.

Disadvantageously, both the solutions described above, in the same way as the other similar ones know in the prior art, are linked to the presence of at least two webs of material for making a pair of flaps having a predetermined orientation.

Moreover, it should be noted that the absorbent sanitary articles mentioned above mainly require two pairs of flaps having an opposite orientation to each other and, generally, made with separate material.

In light of that, it is evident that the prior art methods require the manufacturer to have production lines having at least four feed webs, each to be manipulated by suitable rollers or drums, which considerably complicates the structure of the line, making them expensive and bulky (as well as being more prone to faults).

### DISCLOSURE OF THE INVENTION

For this reason, the aim of this invention is to provide a method for making absorbent sanitary articles which can overcome the above-mentioned drawbacks of the prior art.

More specifically, the aim of this invention is to provide a method for making absorbent sanitary articles which is able to reduce to the maximum extent the number of webs necessary for application of the flaps on the supporting web (that is, on the chassis).

Moreover, the aim of this invention is to provide a method for making absorbent sanitary articles which is simple and easy to implement also in the prior art production lines.

These aims are achieved by a method for making absorbent sanitary articles comprising the steps described in one or more of the subsequent claims, and in particular:

preparing at least one continuous web of material having longitudinal edges parallel to each other;

preparing at least a second continuous web of material having longitudinal edges parallel to each other;

cutting the first web in such a way as to divide it into a plurality of portions positioned one after another;

rotating and/or translating at least part of the portions so as to associate them in pairs for defining a succession of pairs of portions;

connecting the portions (preferably of the pairs) to the second web;

According to one aspect of the invention, the cutting step is performed completely upstream of the connecting step and in such a way as to define, on the first web, an alternating succession of first, second, third and fourth portions, having respective orientations different from each other and each equipped with a first and a second side parallel to each other, defining part of the longitudinal edges of the first web.

In other words, the first web is cut in such a way as to divide it into a sequence of portions, each defined by a succession of a first, a second, a third and a fourth portion.

Advantageously, thanks to the method described above, it is possible to make an ordered sequence of pairs of portions (having the same or opposite orientation) starting from an single web of material, preferably elastic material.

This considerably simplifies the structure of the production line, halving the number of drums necessary or at least limiting their axial extension, with considerable advantages in terms of space occupied and inertias (and thus production speed).

Advantageously, the method according to this invention comprises dividing the web (of elastic material) in four types of different portions, that is to say, each having its own orientation, so as to allow, following suitable rotations and/or translations, a sequence of pairs of portions to be made having the same orientation or alternating orientations.

In other words, dividing the web into four separate portions in pairs which can be coupled, the method allows maximum versatility and flexibility in the design of the line and the product.

Preferably, the portions all have same geometry and each of them is mirrored relative to the previous portion and overturned by 180° relative to the next, or vice versa.

In that way, by means of simple rotation or translation operations, performed in the plane of the web, it is possible to couple the portions in such a way as to define, for each portion, two pairs of portions which can have the same orientation or opposite orientation.

Advantageously, in this way, even having only a single production line it is possible to produce a plurality of combinations of flaps and consequently a plurality of articles of different type.

### BRIEF DESCRIPTION OF DRAWINGS

Further features and advantages will become more apparent from the following description of a preferred, non limiting example embodiment of a method for making absorbent sanitary articles as illustrated in the accompanying drawings, in which:

FIG. 1 schematically shows a cutting step of the method for making absorbent sanitary articles according to this invention;

FIGS. 2a, 2b, 2c each show a succession of steps defining respective variants according to a first embodiment of the method according to this invention;

FIG. 3 shows a positioning step according to both the embodiments of FIGS. 2a-2c;

FIGS. 4a and 4b show the result of the positioning step of FIG. 3;

FIG. 5 shows a schematic side view of a device designed to implement the connecting step according to both the embodiments of FIGS. 2a-2c;

FIGS. 6a, 6b, 6c each show a succession of steps defining respective variants according to a second embodiment of the method according to this invention;

FIG. 7 shows a step after those of FIGS. 6a, 6b, 6c;

FIG. 8 is a sequence of steps according to a third embodiment of the method for making absorbent sanitary articles according to this invention;

FIG. 9 shows a step after those of FIG. 8;

FIG. 10 shows an absorbent sanitary article made with the method according to this invention.

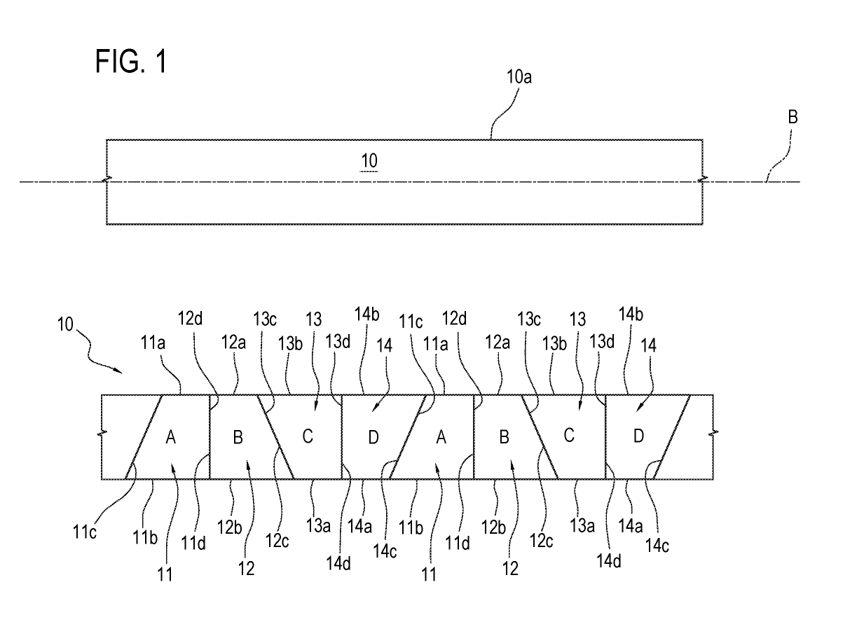

### DETAILED DESCRIPTION OF PREFERRED EMBODIMENTS OF THE INVENTION

With reference to the accompanying drawings, the numeral 1 denotes an absorbent sanitary article made with a method according to this invention and illustrated schematically in FIG. 10.

More specifically, the term “absorbent sanitary article” is used in this text to define a nappy for babies and children or an incontinence pad for adults, or an undergarment equipped with a central absorbent portion and a plurality of side accessory elements.

The accessory elements are preferably flaps or elastic closing elements.

The absorbent article 1 has a substantially rectangular shape extending along a longitudinal axis “A”.

The absorbent articles 1 comprise, in a line along the axis “A”, a front portion 2, a central portion 3 and a rear portion 4.

The absorbent articles 1 comprise an internal absorbent padding, normally made from cellulose fibres, placed inside a soft container defined on one side by a permeable sheet of non-woven fabric, or tope sheet, and, on the other side, by an impermeable sheet of polyethylene, or back sheet.

At the central portion 3, the absorbent articles 1 comprise a recess 5, or leg opening, formed by two arched stretches which are symmetrical relative to the axis “A”.

In this regard, the absorbent article 1 is also equipped with a pair of accessory elements 6, that is flaps, extending transversely to the axis “A”. More specifically, the lateral flaps 6 extend from the rear portion 4 of the absorbent article 1 and are, in use, designed to be placed over respective fastening zones of the front portion 2 in order to close the absorbent article 1 round the wearer's hips.

The lateral flaps 6 generally consist of portions 11, 12, 13, 14 of a continuous web 10 and, preferably, have a surface which is partly covered with an adhesive substance, or provided with other quick fastening means.

The method according to this invention comprises preparing at least a first continuous web 10 of material, preferably material suitable for being used for making separate accessory elements 6 of the absorbent sanitary article 1.

The web 10 extends along a respective direction of unwinding, or operating direction “B,” between two longitudinal edges 10a, 10b parallel to each other.

The first web 10 is then cut in such a way as to divide it into a plurality of portions 11, 12, 13, 14 positioned one after another.

Each portion 11, 12, 13, 14 is shaped to form a flap 6 (or a separate accessory element) of the sanitary article 1.

Each portion 11, 12, 13, 14 is substantially trapezoidal in shape and is equipped with a first side (or smaller base) 11a, 12a, 13a, 14a and a second side (or larger base) 11b, 12b, 13b, 14b positioned opposite and parallel to each other.

These sides are connected to each other by at least an oblique side 11c, 12c, 13c, 14c defining the leg opening 5 of the article 1.

It should be noted that, preferably, the first 11a, 12a, 13a, 14a and the second side 11b, 12b, 13b, 14b each constitute part of the first 10a or second longitudinal edge 10b of the first web 10.

According to one aspect of the invention, the cutting step is performed in such a way as to form, on the first web 10, an alternating succession of first 11, second 12, third 13 and fourth portions 14, having respective orientations different from each other.

Each first 11, second 12, third 13 and fourth portion 14 is equipped with the first 11a, 12a, 13a, 14a and second side 11b, 12b, 13b, 14b parallel to each other.

It should be noted that the expression “alternating succession” means that two portions 11, 12, 13, 14 of the same type are alternated by the presence of the other three.

Thus, by way of example, two first portions 11 are separated from each other by a second 12, a third 13 and a fourth portion 14.

Similar considerations may be made relative to the second 12, third 13 and fourth portions 14.

Thus, the alternating succession is of an ordered type, since the sequence for each type of portion 11, 12, 13, 14 is constant.

In other words, the first web 10 is cut in such a way as to divide it into a sequence of portions 15, each defined by an ordered succession of a first 11, a second 12, a third 13 and a fourth portion 14.

Preferably, the portions 11, 12, 13, 14 all have same geometry and each of them is mirrored relative to the previous portion and overturned by 180° in the plane of the first web 10 relative to the next, or vice versa.

The portions are substantially equal and the difference between each type is defined by its orientation.

More specifically, each first portion 11 has an orientation, measured as an angle in the plane in which it lies, different from the second 12, third 13 and fourth portions 14.

Each second portion 12 has an orientation, measured as an angle in the plane in which it lies, different from the first 11, third 13 and fourth portions 14.

Each third portion 13 has an orientation, measured as an angle in the plane in which it lies, different from the first 11, second 12 and fourth portions 14.

Each fourth portion 14 has an orientation, measured as an angle in the plane in which it lies, different from the first 11, second 12 and third portions 13.

Preferably, as mentioned above, the first sides 11a, 12a, 13a, 14a are shorter than the second sides 11b, 12b, 13b, 14b.

More specifically, so as to allow the article 1 to be made with the elimination of rejects (or waste), preferably, the portions 11, 12, 13, 14 have the shape of a right-angled trapezium.

In other words, in addition to the first side 11a, 12a, 13a, 14a, the second side 11b, 12b, 13b, 14b and the oblique side 11c, 12c, 13c, 14c, the portions have a third side 11d, 12d, 13d, 14d at right angles to the first side 11a, 12a, 13a, 14a and to the second side 11b, 12b, 13b, 14b.

In other words, the third side 11d, 12d, 13d, 14d is oriented at right angles to the operating direction “B”.

Preferably, the third side 11b, 12b, 13b, 14b connects each first portion 11 to the second 12 and each third portion 13 to the fourth 14.

It should be noted that each portion 15 comprises a portion, symmetrical relative to the operating direction “B”, symmetrical relative to third side 11d, 12d, 13d, 14d and a portion overturned relative to it by 180°.

For example, in the embodiment illustrated:

the second portion 12 is symmetrical of the first 11 relative to the third side 11c;

the third portion 13 is symmetrical of the first 11 relative to the operating direction “B”;

the fourth portion 14 corresponds to the first 11 overturned by 180°.

Similar considerations may be made for each of the portions 11, 12, 13, 14 of a single portion 15.

For this reason, the first 11 and the second portion 12 define, on the first web 10, a first isosceles trapezium 16a.

The third sides 11d, 12d of the first and the second portion, adjacent with each other, thus define an axis of symmetry of the first isosceles trapezium 16a.

Similarly, the third 13 and the fourth portion 14 define, on the first web 10, a second isosceles trapezium 16b.

The third sides 13d, 14d of the third and the fourth portion, adjacent with each other, define an axis of symmetry of the second isosceles trapezium 16b.

More specifically, the first portion 11 with the second 12 and the third portion 13 with the fourth 14 define, on the first web 10, a pair of substantially isosceles trapeziums 16a, 16b offset, that is, rotated, between each other by 180°.

After cutting, the method comprises a step of rotation and/or translation of at least part of the portions 11, 12, 13, 14 so as to form a succession of pairs of portions 18, 19.

Preferably, the portions 11, 12, 13, 14 are rotated and/or translated to associate them in pairs.

More specifically, during the rotation and/or translation step the portions are coupled in such a way that, alternatively, in each pair 18, 19 the first 11a, 12a, 13a, 14a or the second sides 11b, 12b, 13b, 14b are opposite each other.

In other words, in a first embodiment, in each pair the first side 11a, 12a, 13a, 14a of a portion 11, 12, 13, 14 faces (or abuts) the first side of the other portion.

In a second embodiment, in each pair the second side 11b, 12b, 13b, 14b of a portion 11, 12, 13, 14 faces the second side of the other portion.

In the first embodiment, the rotation and/or translation step can be performed in various ways, both mainly with rotations and mainly with translations.

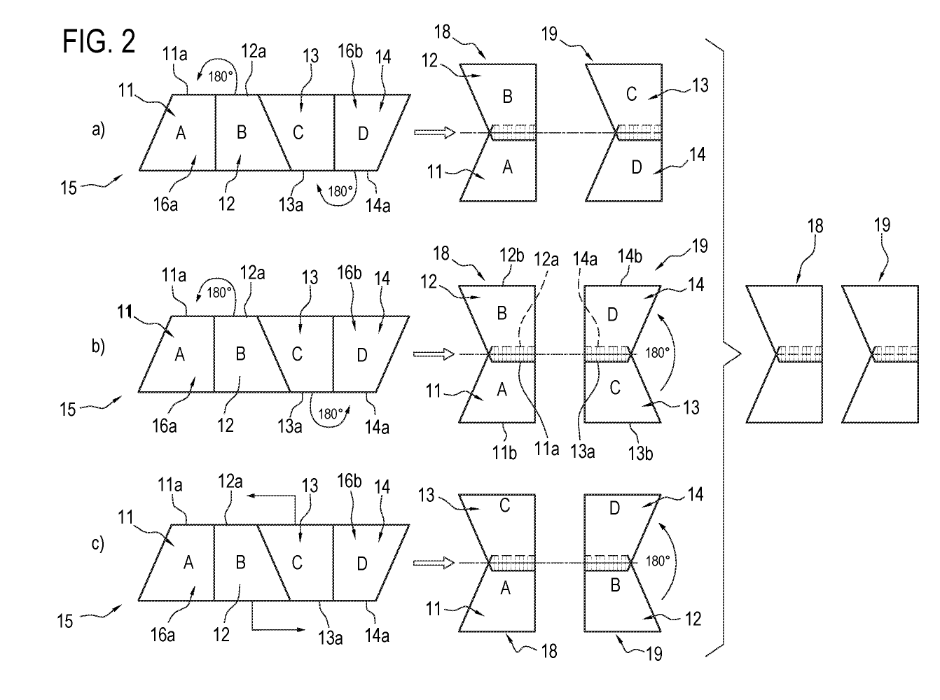

With reference to FIGS. 2a, 2b, the rotation and/or translation step comprises a relative rotation of 180° between each first 11 and second portion 12, in such a way that the respective first sides 11a, 12a are facing or abutting with each other so as to define a first pair 18 of portions.

Moreover, in this embodiment there is a further relative rotation by 180° between each third 13 and fourth portion 14, in such a way that the respective first sides 13a, 14a are facing or abutting with each other in order to define a second pair of portions 19.

The expression “relative rotation of 180°” means that, in each pair 18, 19, the sum of the rotations of each of the two portions relative to the other is equal to 180°.

Consequently, in the first pair 18, the rotation can be performed in one of the following ways:

the first portion 11 can be rotated by 180° whilst the second 12 remains angularly stationary;

the second portion 12 can be rotated by 180° whilst the first 11 remains angularly stationary;

the first 11 and the second portion 12 can be rotated towards each other in such a way that the sum of the angles travelled is equal to 180°.

Similarly, in the second pair 19, the rotation can be performed in one of the following ways:

the third portion 13 can be rotated by 180° whilst the fourth 14 remains angularly stationary;

the fourth portion 14 can be rotated by 180° whilst the third 13 remains angularly stationary;

the third 13 and the fourth portion 14 can be rotated towards each other in such a way that the sum of the angles travelled is equal to 180°.

In the preferred embodiment, and with reference to the accompanying drawings, the rotation and/or translation step comprises rotating the second portion 12 in an anticlockwise direction on the first 11 for making the first pair 18 and the fourth portion 14 in a clockwise direction on the third 13 for making the second pair 19.

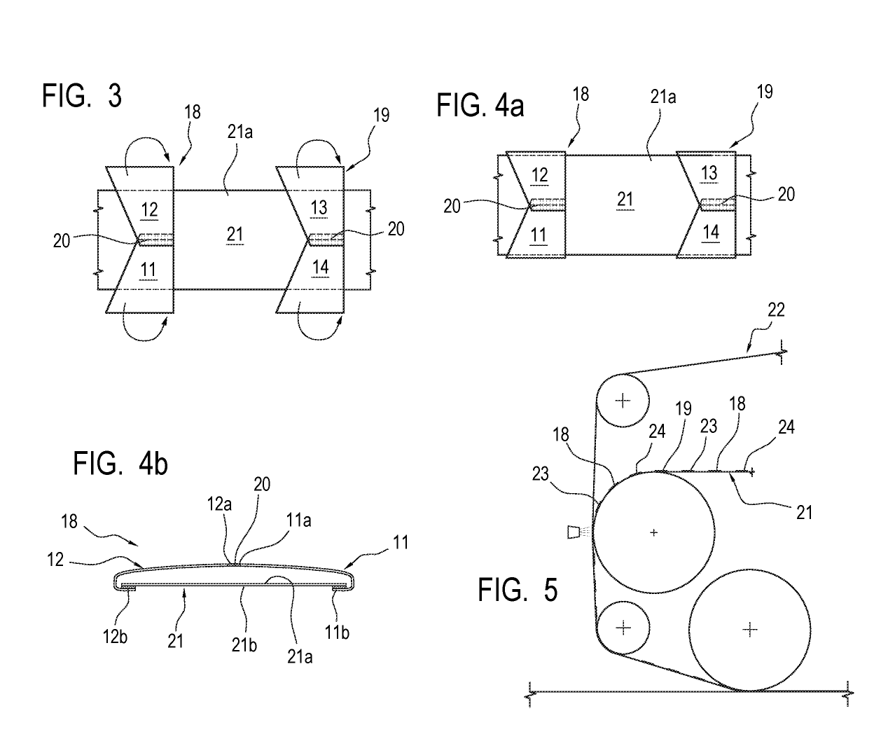

Alternatively, with reference to FIG. 3, the rotation and/or translation step comprises rotating the second portion 12 on the first 11 in an anticlockwise direction for making the first pair 18 and the third portion 13 in the same direction (anticlockwise) on the fourth 14 for making the second pair 19; after that, there is preferably a further rotation by 180° of the second pair 19 in such a way as to make it concordant with the first 18.

Clearly, the references “clockwise direction” and “anticlockwise direction” are used for the sake of descriptive simplicity with reference to the accompanying drawings, but they might be reversed or varied according to the real positioning of the web and/or observer falling within the spirit of this invention.

In a further alternative, shown in FIG. 2c, the individual portions 11, 12, 13, 14 do not undergo any (relative) rotation.

More specifically, in this embodiment, each portion 11, 12, 13, 14 is connected, following a rigid translation one or both the portions of the pair 18, 19, to the portion of the portion 15 symmetrical to it relative to the operating direction “B”.

In this way, a first 18 and a second pair 19 of portions opposite to each other are obtained.

Subsequently, the second pair 19 obtained in this way is overturned, that is, rotated by 180° in the plane, to make it concordant with the first 18 (or vice versa).

In some embodiments, wherein both the pairs 18, 19 are positioned on a same article 1, the overturning (or further rotation) of the second pair 19 is not performed, keeping the first pair 18 oriented in the opposite fashion to the second 19.

Preferably, in all the embodiments of FIGS. 2a, 2b, 2c the method comprises a step of abutting the first side 11 a of each first portion 11 with the first side 12a of the second portion 12 and of the first side 13a of each third 13 with the first side 14a of the fourth portion 14.

Subsequently or simultaneously, these first sides are fixed to each other using constraining means 20.

Following this step, the first side 11a of the first portions 11 is fixed to the first side 12a of the second portions 12.

Similarly, the first side 13a of the third portions 13 is fixed to the first side 14a the fourth portions 14.

It should be noted that, preferably, the constraining means 20 are defined by a layer of adhesive process material or by ultrasound sealing.

The term “adhesive process material” is used to mean an adhesive which is able to keep connected the first sides 11a, 12a, 13a, 14a during the making of the absorbent article 1, but which has limited sealing in such a way as to allow the detachment between the two portions during the use of the article 1.

Also in the second embodiment of the method according to this invention, wherein in each pair the second side 11b, 12b, 13b, 14b of a portion 11, 12, 13, 14 faces (or abuts) the second side of the other portion, the configuration of the first 18 and of the second pair 19 can be obtained with various processes, both mainly by rotation and mainly by translation.

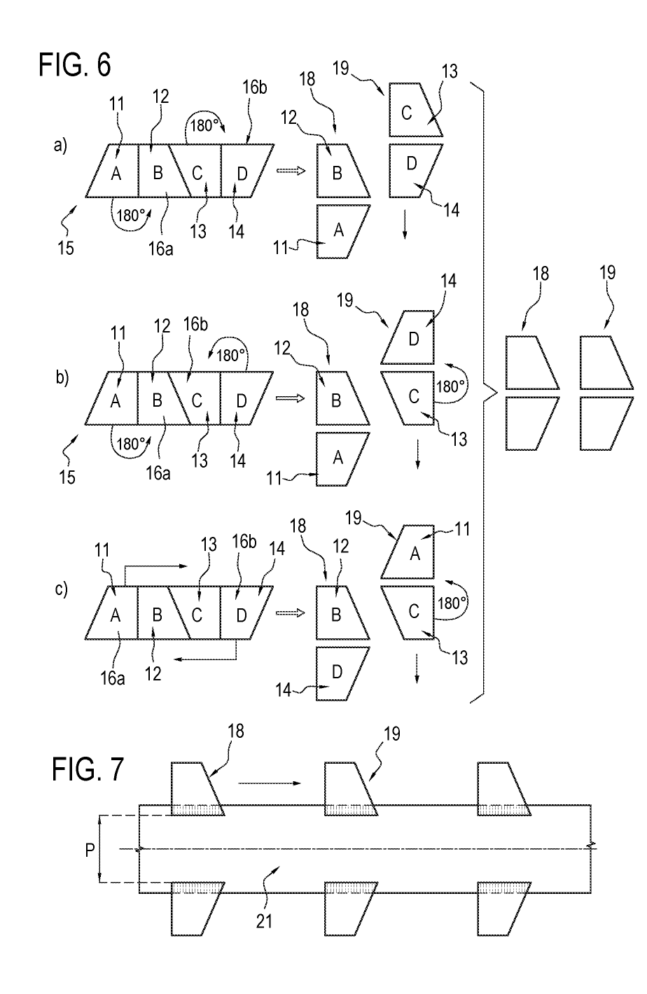

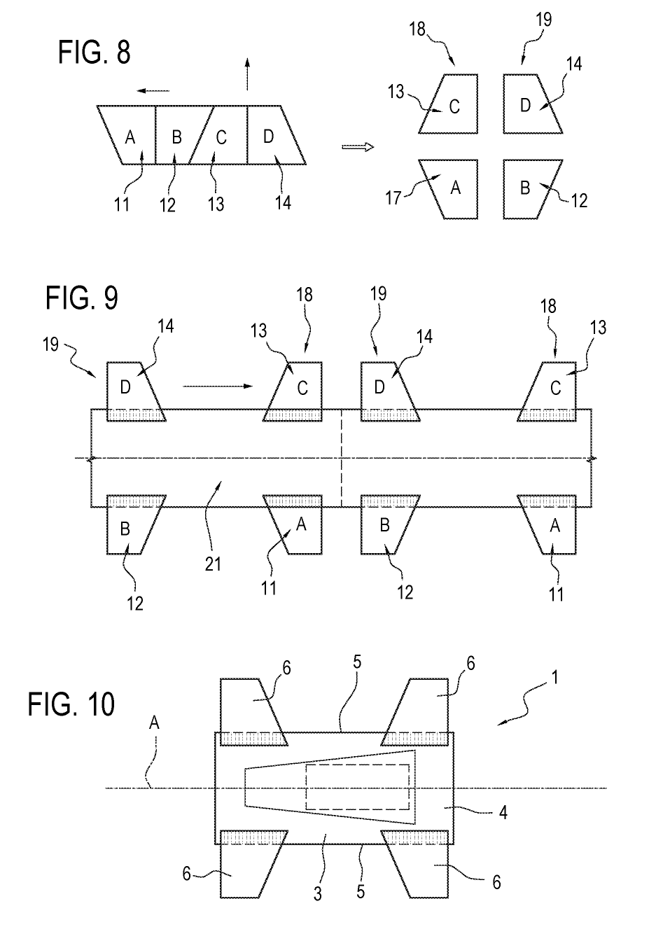

With reference to FIGS. 6a, 6b, the rotation and/or translation step comprises a relative rotation of 180° between each first 11 and second portion 12, in such a way that the respective second sides 11b, 12b are facing each other so as to define the first pair 18 of portions.

Similarly, there is also a relative rotation by 180° between each third 13 and fourth portion 14, in such a way that the respective second sides 13b, 14b face each other so as to define the second pair 19 of portions.

Alternatively, with reference to FIG. 6c, the rotation and/or translation step comprises a rigid translation of the portions 11, 12, 13, 14.

More specifically, each portion 11, 12, 13, 14 faces (that is, is transversally aligned to the operating direction “B”), following a rigid translation of one or both the portions of the pair 18, 19, to the portion of the portion 15 symmetrical to it relative to the operating direction “B”.

Depending on the desired shape of the absorbent article 1, the second pair 19 of portions may undergo a rotation by 180° aimed at making it concordant with the first 18 (or vice versa), or it may be maintained with an orientation opposite to it.

It should be noted that, unlike the first embodiment, in the second embodiment described here the pairs of portions 18, 19 are defined by two portions 11, 12, 13, 14 aligned with each other (transversely to the operating direction “B”) and spaced at a predetermined interval “p”.

In the preferred embodiments, the method comprises performing an aligning step between the first pair 18 and the second pair 19 of portions along a common middle axis (parallel to the operating direction “B”).

Preferably, this step is performed by translating the first pair 18 and/or the second pair 19 of portions along a direction at right angles to the operating direction “B” (that is, to the longitudinal axis of extension of the first web 10).

Following the rotation and/or translation step, the method comprises connecting each pair 18, 19 of portions to a second web 21 of material.

It should be noted that the pair 18, 19 of portions could be formed and then applied to the second web 21, as it could be formed directly during the application of the individual portions 11, 12, 13, 14 on the second web 21 (for example in the embodiments illustrated in FIGS. 6a, 6b, 6c).

The second web 21 is in turn fed along a direction of movement parallel to its direction of extension and has a first face 21a and a second face 21b opposite one another (thus, the second face 21b is opposite the first 21a).

With reference to the first embodiment, wherein the portions 11, 12, 13, 14 are connected to each other at the first side 11a, 12a, 13a, 14a, preferably the pairs 18, 19 of portions (preformed) are superposed on the first face 21a of the second web (in use, outer face).

This superposing is performed in such a way that the second sides 11b, 12b, 13b, 14b of the portions of each pair 18, 19 protrude laterally to the second web 21 (that is, they extend beyond the edge).

In this regard, the method comprises subsequently (or simultaneously) folding the second sides 11b, 12b, 13b, 14b in such a way as to abut them to the second face 21b of the second web 21.

The second sides 11 b, 12b, 13b, 14b are then joined (that is, fixed) to the second face 21b of the second web 21. The fixing is performed preferably by means of a layer of adhesive material or by ultrasound sealing.

Preferably, also, the same method is performed with a fourth web, not illustrated, of material designed to form the flaps 6 of the absorbent sanitary article 1.

This fourth web is cut in such a way as to form respective portions, which are rotated and/or translated in the same way as the portions 11, 12, 13, 14 of the first web 10 to make respective pairs 23, 24.

Preferably, the pairs 23, 24 of the fourth web have an opposite orientation relative to the first 18 and second pair 19.

More preferably, the pairs 23, 24 have the oblique side facing that of the portions of the first 18 and second pair 19.

Thus, before, simultaneously or after applying the first 18 and the second pair 19 to the second web 21, the pairs 23, 24 are also applied and fixed to it.

Therefore, preferably, along the extension of the second web 21 an alternating sequence is formed of a first pair 18, a pair 23, a second pair 19 and a pair 24 (FIG. 5).

It should be noted that, in this embodiment (FIG. 5), the second face 21b of the second web 21 is designed to be connected to a third web 22.

In the absorbent article 1, the second web 21 is known as “top sheet” and is made of non-woven fabric.

On the contrary, the third web 22 is known as “back sheet” and it is made from impermeable material.

With reference to FIG. 5, the method preferably comprises abutting and joining the third continuous web 22 to the second face 21b of the second web 21.

This step is performed after the step of joining the second sides 11b, 12b, 13b, 14b of the portions to the second face 21b.

It should be noted that, preferably, the method also comprises introducing at least a portion of absorbent material (composite) between the first web 21 and the second web 22.

With reference to the second embodiment, wherein the portions 11, 12, 13, 14 are facing each other at the second side 11b, 12b, 13b, 14b, the method preferably comprises fixing the second sides to the second web 21, in the proximity of its longitudinal edges.

In that case, the second web 21 may, without distinction, be impermeable (back sheet) or made from non-woven fabric, or the like (top sheet).

The invention achieves the preset aims and provides major advantages.

In effect, the implementation of a method wherein the web of elastic material for making the flaps is cut into sequences of four portions separate from each other allows the making of the flaps (or at least the pair of front or rear flaps of the nappy) to be completed starting from a single continuous web and minimising rejects or waste.

1. A method for making absorbent sanitary articles, comprising the steps of:
preparing at least one continuous web of material having longitudinal edges parallel to each other;
preparing at least one second continuous web of material;
cutting the first web in such a way as to divide it into a plurality of portions positioned one after another;
rotating and/or translating at least some of the portions so as to associate them in pairs for defining a plurality of pairs of portions;
connecting the portions to the second web;
wherein the cutting step is performed completely before the connecting step and in such a way as to form, on the first web, an alternating succession of first, second, third and fourth portions having the shape of a right-angled trapezium, having respective orientations different from each other and each equipped with a first and a second side parallel with each other, defining part of the longitudinal edges of the first web, wherein the first sides are shorter than the second sides.

2. The method according to claim 1, wherein the first and second portion and the third and fourth portion define, on the first web, a pair of substantially isosceles trapeziums offset from each other by 180°.

3. The method according to claim 2, wherein the portions all have a third side which connects the first portion to the second and the third portion to the fourth; the third sides defining an axis of symmetry of the respective substantially isosceles trapezium.

4. The method according to claim 1, wherein the step of rotating and/or translating at least part of the portions comprises a relative rotation of 180° between each first and second portion, in such a way that the respective first or second sides are facing or abutting with each other so as to define a first pair of portions.

5. The method according to claim 4, wherein the step of rotating and/or translating at least part of the portions comprises a relative rotation of 180° between each third and fourth portion, in such a way that the respective first or second sides are facing or abutting with each other so as to define a second pair of portions.

6. The method according to claim 4, wherein the steps of relative rotation of 180° between the first and the second portions and between the third and the fourth portions comprise abutting the first sides with each other and, subsequently or simultaneously, fixing the first side of the first portions to the first side of the second portions and the first side of the third portions to the first side of the fourth portions using constraining means.

7. The method according to claim 6, wherein the constraining means are defined by a layer of adhesive material or by heat sealing.

8. The method according to claim 6, wherein the step of connecting the first and the second pair of portions to the second web comprises the following sub-steps:
superposing each pair of portions on a first face of the second web in such a way that the second sides of the portions of each pair protrude laterally to the second web;
folding the second sides in such a way as to abut them on a second face of the second web, opposite the first;
joining the second sides to the second face of the second web.

9. The method according to claim 8, wherein it comprises a step for abutting and joining a third continuous web to the second face of the second web performed downstream of the step for joining the second sides of the portions of the second face.

10. The method according to claim 1, wherein relative rotation of 180° between the first and the second portions is performed with a direction of rotation opposite to that of the relative rotation of 180° between the third and the fourth portions.

11. The method according to claim 1, wherein it comprises a step of alignment between the first pair and the second pair of portions along a shared median axis.

12. The method according to claim 11, wherein the aligning step is performed by translating the first pair and/or the second pair of portions along a direction at right angles to a longitudinal axis of extension of the first web.

13. The method according to claim 1, wherein:
each first portion has an orientation, measured as the angle in the relative plane in which it lies, which is different from the second, third and fourth portions;
each second portion has an orientation, measured as the angle in the relative plane in which it lies, which is different from the first, third and fourth portions;
each third portion has an orientation, measured as the angle in the relative plane in which it lies, which is different from the first, second and fourth portions;
each fourth portion has an orientation, measured as the angle in the relative plane in which it lies, which is different from the first, second and third portions.
